(19) United States
(12) Patent Application Publication
(10) Pub. No.: US 2024/0099961 A1
(43) Pub. Date: Mar. 28, 2024

(54) FERMENTED EXTRACT OF LONGOZA SEEDS

(71) Applicant: L V M H RECHERCHE, Saint-Jean-De-Braye (FR)

(72) Inventors: Lorène GOURGUILLON, Saint-Jean-De-Braye (FR); Patrick CHOISY, Saint-Jean-De-Braye (FR); Carine NIZARD, Saint-Jean-De-Braye (FR); Olivier JEANNETON, Saint-Jean-De-Braye (FR)

(73) Assignee: L V M H RECHERCHE, Saint-Jean-De-Braye (FR)

(21) Appl. No.: 18/268,679

(22) PCT Filed: Dec. 17, 2021
(86) PCT No.: PCT/FR21/52388

§ 371 Date: Jun. 21, 2023

(30) Foreign Application Priority Data
Dec. 30, 2020 (FR) FR2014275

(51) Int. Cl. A61K 8/9794; A61K 8/67; A61K 36/906; A61Q 19/08

(52) U.S. Cl. CPC A61K 8/9794; A61K 8/678; A61K 36/906; A61Q 19/08; A61K 2236/19; A61K 2800/522; A61K 2800/85

(57) ABSTRACT

The present invention relates to a fermented extract of Longoza seeds, as well as to the preparation method thereof and to cosmetic compositions containing same. The fermented extract of Longoza seeds can in particular be used as an active ingredient to promote and/or stimulate epidermal renewal, desquamation of the skin, vitality of the skin cells, and/or cohesion of the skin cells, and/or to prevent and/or reduce the signs of skin ageing, in particular loss of firmness, loss of elasticity, loss of density, appearance of wrinkles and/or fine lines, dryness of the skin, alteration of the skin's barrier function, alteration of the skin's suppleness and/or alteration of the homogeneity or radiance of the complexion.

### FIELD OF THE INVENTION

The present invention relates to the field of cosmetics and, in particular, to a fermented extract of Longoza seeds, as well as the method of fermentation enabling it to be obtained, a cosmetic composition containing said fermented extract of Longoza seeds, and the use thereof in a cosmetic method for care and/or make-up of keratin materials.

### PRIOR ART

It is known that ageing is induced by endogenous factors (e.g. stress, hormones, etc.) and exogenous factors (e.g. temperature, climate, UV radiation, atmospheric pollution, cigarette smoke, etc.) leading to a slow down and/or an alteration of various cellular functions. In the case of the skin, ageing is manifest by various clinical signs, notably by a reduction in epidermal renewal, an alteration of the cutaneous barrier, a loss of firmness and elasticity of the skin, a reduction in skin thickness, the appearance of wrinkles or fine lines, the appearance of a less homogeneous complexion, and/or a dryer skin.

There is therefore a constant need to find agents capable of preventing and/or reducing the signs of skin ageing, notably enabling the combating of stress and/or enabling stimulating and/or promoting of skin renewal, as well as the vitality of skin cells.

Unexpectedly, the Applicant has discovered the ‘anti-ageing’ effects of a fermented extract of Longoza seeds in vitro on a skin model. Indeed, the fermented extract of Longoza seeds can advantageously improve the structure of the skin, which becomes notably thicker, with a very clear basal organisation and structured dermoepidermal junction. Furthermore, the fermented extract of Longoza seeds can advantageously increase the expression of various proteins involved in the mechanical solidity of the skin, as well as its renewal and its elasticity, such as elafin, desmoglein 1, loricrin, transglutaminase and ZO-1, ultimately promoting cellular integrity and homoeostasis of skin cells. These effects therefore make it possible to envisage the use of this fermented extract of Longoza seeds in order to prevent and/or reduce the signs of skin ageing. The fermented extract of Longoza seeds is used, in particular, according to the invention as a cosmetic active ingredient intended to promote and/or stimulate epidermal renewal, desquamation of the skin, vitality of the skin cells, and/or cohesion of the skin cells, and/or to prevent and/or reduce the signs of skin ageing, in particular loss of firmness, loss of elasticity, loss of density, appearance of wrinkles and/or fine lines, dryness of the skin, alteration of the skin's barrier function, alteration of the skin's suppleness and/or alteration of the homogeneity or radiance of the complexion.

### DISCLOSURE OF THE INVENTION

The first object of the invention thus concerns a fermented extract of Longoza seeds comprising a quinic acid content between 0.05 and 2% and an acetic acid content between 5 and 15%, by weight compared with the weight of dry extract of the fermented extract.

Another object of the invention relates to a method for fermenting Longoza seeds, comprising the following steps:
a) incubating a consortium of microorganisms in a solution comprising a carbohydrate at a temperature between 17 and 38° C. for 1 to 10 days in order to obtain a culture of microorganisms,
   said consortium comprising at least one lactic acid bacterium belonging to the genus Lactobacillus or Pediococcus, at least one yeast belonging to the genus Saccharomyces, Schyzosaccharomyces or Torulaspora, and at least one acetic acid bacterium belonging to the genus Acetobacter, Gluconobacter or Komagataeibacter;
b) grinding the Longoza seeds, in order to obtain a powder composed of particles having an average size between 100 μm and 1 mm;
c) adding said powder to a fermentation medium comprising the culture of microorganisms in order to obtain a fermentation mixture, said fermentation medium comprising a carbohydrate chosen from sucrose, glucose, fructose, molasses or a mixture of one or more thereof,
d) incubating the fermentation mixture at a temperature between 25 and 35° C. for 7 to 13 days;
e) filtering the fermentation mixture in order to obtain a fermented extract of Longoza seed.

Also, another object of the invention relates to a fermented extract of Longoza seeds obtained by the method as described according to the invention.

The invention also relates to a cosmetic composition comprising, in a physiologically acceptable medium, a fermented extract of Longoza seeds.

Another object of the invention relates to a cosmetic method for care and/or make-up of keratin materials, in particular the skin and/or lips, comprising the application on said keratin materials, of at least one layer of cosmetic composition as described according to the invention.

Finally, the invention relates to the cosmetic use of a fermented extract of Longoza seeds according to the invention as an active ingredient to promote and/or stimulate epidermal renewal, desquamation of the skin, vitality of the skin cells, and/or cohesion of the skin cells, and/or to prevent and/or reduce the signs of skin ageing, in particular loss of firmness, loss of elasticity, loss of density, appearance of wrinkles and/or fine lines, dryness of the skin, alteration of the skin's barrier function, alteration of the skin's suppleness and/or alteration of the homogeneity or radiance of the complexion.

### DESCRIPTION OF THE FIGURES

FIG. 1 Effect of the fermented extract of Longoza seeds on the expression of loricrin

FIG. 2 Effect of the fermented extract of Longoza seeds on the expression of filaggrin

FIG. 3 Effect of the fermented extract of Longoza seeds on the expression of elafin

FIG. 4 Effect of the fermented extract of Longoza seeds on the expression of kallikrein 5

FIG. 5 Effect of the fermented extract of Longoza seeds on the expression of desmoglein 1

FIG. 6 Effect of the fermented extract of Longoza seeds on the expression of transglutaminase

FIG. 7 Effect of the fermented extract of Longoza seeds on the expression of ZO-1

FIG. 8 Histological analysis of the effect of the fermented extract of Longoza seeds on a model of the skin

FIG. 9 Effect of the fermented extract of Longoza seeds on the elastic modulus of the skin

### DETAILED DESCRIPTION OF THE INVENTION

Aframomum angustifolium or Longoza

Originating from Madagascar, Longoza is a fruit of Aframomum angustifolium, a plant of the ginger family. This plant is characterised through an exceptional regeneration by the root, since it grows even after cutting and burning. It is one of the pioneer plants which grow again on ground that is burnt during forest fires on Madagascar.

In the context of the present invention, the fermented extract of Aframomum angustifolium or Longoza is obtained from the seeds of the Aframomum angustifolium or Longoza plant. Although the expression “Longoza seeds” is used here by way of simplification, this obviously refers interchangeably to seeds of Aframomum angustifolium or Longoza.

Fermented Extract of Longoza Seeds

Longoza seeds are used in a fermentation method in order to generate a fermented extract of Longoza seeds, the object of the present invention.

It is specified that in the present description, the expression “fermented extract” designates the product coming from the fermentation method involving fermentation with or without filtration, therefore both with and without the microbial biomass of the fermentation medium, as well as any product having undergone additional steps, such as a formulation step (for example, by addition of a preservative).

According to a particular embodiment, the fermented extract of Longoza seeds according to the invention is in the form of an aqueous solution of fermented extract comprising the fermented dry extract, water and a preservative. The fermented extract in the form of an aqueous solution is also termed ‘raw material’ in the description, as opposed to ‘dry matter’ or fermented dry extract.

According to a particular embodiment, the aqueous solution of fermented extract comprises the fermented extract with an active content (dry matter) ranging from 0.5 to 3% by weight, notably 1 to 2% by weight and in particular 1.2 to 1.7% by weight of active material compared with the total weight of raw material (aqueous solution of fermented extract), water and a preservative, preferably glycerin. The water content generally ranges from 20 to 40% by weight, notably from 25 to 30%, in particular from 27 to 29% by weight compared with the total weight of raw material (aqueous solution of fermented extract) and the preservative content (e.g. glycerin) will generally range from 60 to 80% by weight, notably from 65 to 75% by weight, in particular 70% by weight compared with the total weight of raw material (aqueous solution of fermented extract).

Thus, the invention also relates to a fermented extract of Longoza seeds in the form of a solution comprising 1 to 2% by weight of active material of fermented extract of Longoza seeds, 65 to 75% by weight glycerin and 25 to 30% by weight water.

Preferably, the aqueous solution of fermented extract, otherwise termed ‘raw material’ in the characterisation illustrated below, comprises 1.2 to 1.65% active material (dry matter) of fermented extract, 70% glycerin and 28.35 to 28.8% water compared with the total weight of raw material (aqueous solution of fermented extract). According to a particular embodiment, the aqueous solution of fermented extract, otherwise termed ‘raw material’ in the characterisation illustrated below, comprises 1.2 to 1.35% active material (dry matter) of fermented extract, 70% glycerin and 28.65 to 28.8% water compared with the total weight of raw material (aqueous solution of fermented extract). According to another particular embodiment, the aqueous solution of fermented extract, otherwise termed ‘raw material’ in the characterisation illustrated below, comprises 1.35 to 1.65% active material (dry matter) of fermented extract, 70% glycerin and 28.35 to 28.65% water compared with the total weight of raw material (aqueous solution of fermented extract).

According to a particular embodiment, the fermented extract of Longoza seeds has a quinic acid content greater than 0.005%, preferably between 0.005 and 10%, and an acetic acid content greater than 0.5%, preferably between 0.5 and 25%, by weight compared with the weight of dry extract (dry matter) of the fermented extract. Preferably, the fermented extract of Longoza seeds has a quinic acid content between 0.01 and 5%, more preferably between 0.05 and 2% by weight compared with the weight of dry extract of the fermented extract. Preferably, the fermented extract of Longoza seeds has an acetic acid content between 1 and 20%, more preferably between 5 and 15% by weight compared with the weight of dry extract of the fermented extract.

The fermented extract of Longoza seeds can comprise other organic acids, such as citric acid, gluconic acid, lactic acid, malic acid, and succinic acid. Also, the fermented extract of Longoza seeds can notably have:
a citric acid content between 0.01 and 5%, preferably between 0.05 and 2% more preferably between 0.1 and 1%, still more preferably between 0.2 and 0.4% by weight compared with the weight of dry extract of the fermented extract,
a gluconic acid content greater than 0.5%, preferably between 0.5 and 25%, more preferably between 1 and 20%, still more preferably between 1 and 15% by weight compared with the weight of dry extract of the fermented extract,
a lactic acid content between 0.001 and 10%, preferably between 0.005 and 5%, more preferably between 0.01 and 2% more preferably between 0.01 and 1.5% by weight compared with the weight of dry extract of the fermented extract,
a malic acid content between 0.001 and 5%, preferably between 0.005 and 1%, more preferably between 0.008 and 0.5% by weight compared with the weight of dry extract of the fermented extract, and/or
a succinic acid content between 0.001 and 5%, preferably between 0.005 and 1%, more preferably between 0.01 and 0.05% by weight compared with the weight of dry extract of the fermented extract.

Preferably, the fermented extract of Longoza seeds does not comprise glucuronic acid or propionic acid.

The fermented extract of Longoza seeds can also comprise one or more preservatives. The term “preservative” shall mean a compound which has the purpose of preserving the composition of the extract or the cosmetic containing it from any physical, chemical and/or microbial alteration. Preferably, at least one preservative is chosen from butylene glycol, glycerin, propylene glycol, sodium benzoate benzoate and potassium sorbate, more preferably glycerin. The fermented extract can notably comprise one or more preservatives at a final concentration between 60 and 80%, preferably between 65 and 75%, more preferably 70% by weight compared with the total weight, preferably at least one of the preservatives described above, chosen from butylene glycol, glycerin and propylene glycol.

Fermentation Method

This fermented extract of Longoza seeds is obtained by a fermentation method comprising the steps of:
a) incubating a consortium of microorganisms in a solution comprising a carbohydrate and optionally also an aqueous extract of tea leaves at a temperature between 17 and 38° C., in order to obtain a culture of microorganisms;
b) grinding seeds of Aframomum angustifolium or Longoza in order to obtain a fine powder;
c) adding said powder to a fermentation medium comprising the culture of microorganisms and a carbohydrate, in order to obtain a fermentation mixture;
c) incubating the fermentation mixture at a temperature between 12 and 42° C. for 2 to 20 days;
d) filtering the fermentation mixture, in order to obtain a fermented extract of Longoza seeds; and, optionally,
e) adding at least one preservative.

The term “fermentation method” shall mean a method enabling a fermented extract of Longoza seeds to be obtained by culturing a consortium of microorganisms as defined here, in a medium containing a carbohydrate and enabling their growth. The fermentation can take place under discontinuous culture conditions (“batch”), continuous culture conditions, or continuous/discontinuous conditions (“Fed batch”). The fermentation can be carried out aerobically, micro-aerobically and/or anaerobically.

The term “consortium of microorganism” shall mean an assembly of at least three species of microorganisms which can coexist in a stable and reproducible manner.

Advantageously, the microorganisms can combine complementary activities. By way of non-limiting example, the use or assimilation of a source of carbon by a first species can produce metabolites that can then be metabolised by a second species.

The consortium of microorganisms according to the present invention comprises:
i) at least one microorganism belonging to the group of lactic acid bacteria,
ii) at least one microorganism belonging to the group of yeasts, and
iii) at least one microorganism belonging to the group of acetic acid bacteria.

Preferably, at least one microorganism belonging to the group of lactic acid bacteria is a microorganism of the order Lactobacillales of the family Lactobacillaceae, notably those belonging to the genera Lactobacillus and Pediococcus, such as Lactobacillus plantarum and/or Lactobacillus acidophilus, more preferably L. plantarum.

Preferably, at least one microorganism belonging to the group of yeasts is a microorganism of the order Saccharomycetales of the family Saccharomycetaceae or of the order Schyzosaccharomycetales of the family Schyzosaccharomycetaceae, notably those belonging to the genera Saccharomyces, Schyzosaccharomyces or Torulaspora.

Preferably, at least one microorganism belonging to the group of yeasts is Saccharomyces cerevisiae, Torulaspora delbrueckii, Saccharomyces boulardii and/or Schyzosaccharomyces pombe.

Preferably, at least one microorganism belonging to the group of acetic acid bacteria is a microorganism of the order Rhodospirillales of the family Acetobacteraceae, notably those belonging to the genera Acetobacter, Gluconobacter and Komagataeibacter (also called Gluconacetobacter). The acetic acid bacteria can originate from cider vinegar or unpasteurised wine having a level of acidity of 4% to 5%, preferably a commercial cider vinegar, labelled “Organic farming” according to the European Union definition. Preferably, the addition of acetic acid bacteria to the consortium is made by directly adding an aliquot of vinegar to the composition comprising the other species of microorganisms. In vinegars, alongside the dominant acetic acid bacteria, there is also a complex natural indigenous flora, part of which can cohabit with the added microorganisms. Another part will decline during the establishment of the consortium, in the same way as the symbiotic growth of the assembly of microorganisms forming the consortium prevents the growth of undesirable species, including pathogenic species.

According to a preferred embodiment, the consortium comprises:
at least one lactic bacterium belonging to the genus Lactobacillus or Pediococcus, more preferably chosen from Lactobacillus plantarum and Lactobacillus acidophilus,
at least one yeast belonging to the genus Saccharomyces, Schyzosaccharomyces or Torulaspora more preferably chosen from Saccharomyces cerevisiae, Saccharomyces boulardii, Schyzosaccharomyces pombe and Torulaspora delbrueckii, and
at least one acetic acid bacteria coming from cider vinegar or a wine vinegar, preferably belonging to the genus Acetobacter, Gluconobacter or Komagataeibacter.

Step a): Preparation of a Culture of Microorganisms

In a first step, a), of the method, a culture of microorganisms (otherwise termed a consortium comprising the microorganisms as described above) is prepared. This step advantageously allows the microorganisms to be activated and multiply while establishing a stable and balanced consortium. During this step, the various microorganisms which will be present in the consortium are introduced into a solution comprising a carbohydrate (otherwise called a “nutritive medium”) and optionally also an aqueous extract of tea leaves, in order to generate a microbial suspension. The suspension is then incubated for 1 to 21 days in order to prepare the culture of microorganisms (otherwise termed, the consortium).

The solution comprises at least one carbohydrate, generally one or more mono- or disaccharides. This may be a pure sugar, such as sucrose, glucose or fructose, or else be a transformed or co-produced product, such as molasses. Also, according to a preferred embodiment, the solution comprises a carbohydrate chosen from sucrose, glucose or fructose, molasses, or a mixture of one or more thereof. The carbohydrate is preferably sucrose or a mixture of glucose and fructose. Preferably, at least one carbohydrate is present at a concentration between 20 g/l and 100 g/l, preferably between 50 and 95 g/l of solution. Preferably, the nutritive medium comprises sucrose, or a mixture of glucose and fructose, at a concentration of 50 g/l.

According to a particular embodiment, the solution can further comprise an aqueous extract of tea leaves (Camellia sinensis). Preferably, the aqueous extract is obtained by an infusion for tea leaves, preferably black tea leaves. Preferably, the aqueous extract of tea leaves can be an infusion of black tea prepared with 2 g/l to 50 g/l of tea leaves, more preferably with 5 g/l to 10 g/l of tea leaves.

The tea is prepared by adding dry leaves infused in water at a temperature de 70° C. to 90° C. for 10 to 60 minutes. Preferably, 5 g/l to 10 g/l of tea leaves are used, infused 15 minutes at 80° C. The infusion can be used as is with the leaves or, more conveniently, the aqueous phase is filtered.

The microorganisms can be introduced into the nutritive medium in dry and/or wet form; the level of inoculation can be, in particular, between 10³ CFU/g and 10⁵ CFU/g of solution. The microbial suspension can then be incubated at between 12° C. and 45° C., preferably between 25° C. and 35° C., more preferably at 25° C., 26° C., 27° C., 28° C., 29° C., 30° C., 31° C., 32° C., 33° C., 34° C., or 35° C., still more preferably at 26° C. According to a particularly advantageous embodiment, the incubation takes place in an open reactor, without the need to ensure aseptic conditions.

The suspension can be incubated for 1 to 21 days, preferably for 1 to 10 days, more preferably for 3 to 4 days, still more preferably for 3 days or 4 days. At the end of the incubation period, the total flora in the suspension can be between 10⁴ and 10⁷ CFU/g.

The consortium thus obtained is able to react with a natural substrate, such as Longoza seeds, in aqueous medium. Advantageously, it is capable of directing the extractive fermentation of Longoza seeds, in order to make accessible, and possibly transform, a large diversity of compounds that it contains.

Step b): Preparing the Longoza Seeds

In step b), the Longoza seeds are ground until a fine powder is obtained. The particles of this powder advantageously have an average size between 0.01 and 1 mm, preferably between 100 μm and 1 mm, more preferably between 250 and 500 μm, still more preferably an average size of 500 μm. The seeds are preferably raw. The seeds are preferably dried before being ground.

Step b) can equally be carried out before, at the same time as, or after step a).

Step c): Preparing a Fermentation Mixture

In step c) of the method, the powder of Longoza seeds obtained in step b) is added to a fermentation medium, comprising the culture of microorganisms obtained in step a) as well as a carbohydrate, in order to obtain a fermentation mixture.

The culture of microorganisms is preferably added to the fermentation medium at a rate between 10² CFU/g and 10⁴ CFU/g of fermentation medium. Preferably, the culture of microorganisms is added to the fermentation medium at a concentration between 0.5% and 15% by weight compared with the weight of fermentation medium, preferably between 0.75 and 10%, more preferably 5%.

The Longoza seeds in powder form are preferably added to the fermentation medium at a concentration between 1 and 75 g of seeds per litre of fermentation medium (g/L), preferably between 10 and 60 g/L, more preferably between 12 and 50 g/L, still more preferably 12 g/L or 50 g/L. In other words, the Longoza seeds in powdered form are added to the fermentation medium at a concentration between 0.1 and 7.5%, preferably between 1 and 6%, more preferably between 1.2 and 5%, still more preferably at a concentration of 1.2 or 5% by weight compared with the total volume of the fermentation medium.

The fermentation medium also comprises a carbohydrate, preferably chosen from sucrose, glucose, fructose, molasses or a mixture of one or more thereof.

According to a particular embodiment, the solution can comprise an aqueous extract of tea leaves, as described above. Preferably, the aqueous extract is obtained by an infusion for tea leaves, preferably black tea leaves.

In order to facilitate the dissolving of the carbohydrate in the fermentation medium, the latter can be heated before or after adding the carbohydrate, for example at a temperature between 25° C. and 90° C., preferably 35° C. Preferably, in the case of heating the fermentation medium, this is done before adding the culture of microorganisms or even at a temperature less than or equal to 45° C., preferably less than or equal to 35° C., if after addition of the culture.

Step d): Incubation

In the next step d) of the method, the fermentation mixture obtained in step c) is then incubated in order to ensure fermentation of the Longoza seeds.

The incubation takes place at a temperature between 12° C. and 45° C., preferably between 25° C. and 35° C., more preferably at 25° C., 26° C., 27° C., 28° C., 29° C., 30° C., 31° C., 32° C., 33° C., 34° C., or 35° C., still more preferably at 26° C. The incubation is carried out for a period of 2 to 20 days, preferably for 7 to 13 days, more preferably for 7, 8, 9, 10, 11, 12, or 13 days, still more preferably 10 days.

According to a particular aspect, the incubation takes place in an open reactor, without the need to ensure aseptic conditions. According to a particular aspect, the incubation takes place at a humidity level between 20% and 60%, preferably between 30% and 50%, more preferably 40%.

Step e): Filtration

The fermentation mixture is then filtered during step e). Here, the term “filtration” shall mean a step enabling the solid and liquid fractions to separate. The solid fraction can comprise, in particular, the remaining Longoza seeds and/or microorganisms. Advantageously, the filtration can remove all the microorganisms present in the mixture, in order to obtain a sterile fermented extract of Longoza seeds. The filtration can be carried out using one or more sieves and/or filters. Also, according to an embodiment, step e) corresponds to one or more filtration steps, preferably comprising at least one sieving step and/or at least one sterilising filtration step. Preferably, step e) comprises a sieving step and a sterilising filtration step. Preferably, the sterilising filtration is carried out on a filter having an average pore size between 0.1 μm and 0.2 μm, preferably 0.2 μm. Advantageously, the sieving can remove the powdered debris. Advantageously, a sterilising filtration can quickly and inexpensively remove any cellular debris. Preferably, at least one of the filters is a cellulose filter.

Step f): Concentration and/or Formulation (Optional)

The method described above can obtain a clarified fermented extract of Longoza seeds. However, in some cases, it can be advantageous to carry out one or more additional steps, for example for concentration or formulation. The method according to the invention can thus further comprise at least one additional step subsequent to step e), of concentration or formulation.

In a particular embodiment, the method according to the invention further comprises, after the filtration step f), one or more following steps intended, in particular, to adjust the composition to a particular formulation:
a concentration step; and/or
a formulation step.

Each optional step identified above can be present as a unique additional step of the method, or in combination with one or more other optional steps. The fermentation method preferably comprises a step f) of adding at least one preservative to the fermented extract of Longoza seeds. Said preservative is preferably chosen from butylene glycol, glycerin, propylene glycol, sodium benzoate benzoate and potassium sorbate, more preferably glycerin. In certain cases, at least two preservatives can be added to the fermented extract of Longoza seeds. The preservative is preferably added to the fermented extract of Longoza seeds of step f) at a final concentration between 50 and 80%, preferably between 65 and 75%, more preferably 70%, by weight compared with the total weight of the extract in the form of a solution. When the preservative is added to the fermented extract of Longoza seeds of step e) at a final concentration between 50 and 80%, said preservative is selected from butylene glycol, glycerin and propylene glycol.

The fermented extract of Longoza seeds is preferably kept at approximately 4° C. (4±2° C.) and/or in the dark at the end of filtration step e) or at the end of any other step when an additional step is present after step e). When the method comprises the addition of at least one preservative, the fermented extract of Longoza seeds is preferably kept at ambient temperature.

The fermented extract of Longoza seeds is given the reference under the international nomenclature, INCI: “Water, glycerin, Aframomum angustifolium seed extract, lactobacillus ferment lysate, yeast ferment extract”. This product contains, notably, 1.2 to 1.65% by weight of dry extract of fermented extract of Longoza seeds in a water/glycerin mixture.

Cosmetic Composition

Surprisingly, the inventors have determined that the fermented extract of Longoza seeds has, in vitro, beneficial effects on the skin, in particular increasing the number and/or density of fibroblasts, as well as the expression of various proteins involved in the mechanical solidity of the skin, its renewal and its elasticity, such as elafin, desmoglein 1 and loricrin. Thus, the fermented extract of Longoza seeds can combat the signs of skin ageing connected to age and/or stresses, in particular loss of firmness and/or elasticity and alteration of the barrier function of the skin.

According to the invention, the fermented extract of Longoza seeds is used in a quantity effective for obtaining the desired effect. It can be used as is, or advantageously incorporated in a cosmetic composition that is suitable for topical application on keratin materials.

In particular, the fermented extract of Longoza seeds is used as is or is present in a cosmetic composition.

Also, another object of the invention relates to a cosmetic composition comprising, in a physiologically acceptable medium, a fermented extract of Longoza invention seeds according to the invention.

The term “cosmetic composition” shall mean any composition for cosmetic purposes, i.e. aesthetic purposes, that can be placed in contact with the superficial parts of the human body and more particularly with keratin materials, in particular human skin and/or lips.

The term “physiologically acceptable medium” shall mean any excipient that is suitable for topical use, in contact with keratin materials, without the risk of toxicity, incompatibility, instability and/or allergic response.

The term “keratin materials” according to the invention shall mean the skin and/or its appendages, and more particularly human skin and/or lips. In particular, it involves the skin of the face and/or the neck and/or the body, and the lips.

Keratin materials according to the invention are, in particular healthy keratin materials (“healthy” subjects), i.e. not exhibiting diseases or disorders which would be part of a pathological condition (“unhealthy” subjects, affected by a pathology). In the rest of the description, healthy skin and/or lips or skin and/or lips will be referred to interchangeably.

The fermented extract of Longoza seeds is present in the composition of the invention at a level ranging from 0.0001% to 5%, preferably 0.0005% to 2%, more preferably 0.001% to 1% by weight of raw material (fermented extract solution obtained according to the above-described method) compared with the total weight of the composition. Thus the fermented extract of Longoza seeds is present in the composition of the invention at a level ranging from 0.0000012% to 0.0825%, preferably 0.000006% to 0.033%, more preferably 0.000012% to 0.0165% by weight of active material (fermented extract of Longoza seeds) compared with the total weight of the composition.

The additional ingredients advantageously used in the composition of the invention can be present at a level ranging from 0.0001% to 10%, preferably from 0.001% to 5% by weight (of raw material) compared with the total weight of the composition.

The physiologically acceptable medium generally represents 1 to 99% by weight, compared with the total weight of said composition. The physiologically acceptable medium of the composition according to the invention comprises water and optionally the preservative such as previously defined.

In a particular embodiment, said cosmetic composition comprising a fermented extract of Longoza seeds according to the invention is a composition for care and/or make up of keratin materials, in particular the skin and/or the lips and notably the skin of the face and/or of the neck.

The cosmetic composition of the invention generally further comprises fermented extract of Longoza seeds and the physiologically acceptable medium, one or more cosmetically acceptable excipients from those known to a person skilled in the art, with a view to obtaining a composition for topical application, for example in the form of a milk, cream, pomade, water-in-oil or oil-in-water emulsion, salve, stick, gel, lotion, serum, or else powder.

The cosmetic composition of the invention can also be present in the form of a patch or a mask, notably a mask in the form of a thick cream, or else in the form of a cellulose mask impregnated with fermented extract of Longoza seeds or a composition containing same.

In a preferred embodiment, said composition is in the form of a cream, emulsion, solution, suspension, gel, milk, lotion, or serum. When the composition is in the form of an emulsion, the emulsion can be an oil-in-water emulsion, water-in-oil emulsion or multiple emulsion.

The cosmetic composition of the invention can be in galenic form suitable for topical application on keratin materials, in particular of the skin and/or lips and, in particular, the skin of the face and/or of the neck, comprising the fermented extract of Longoza seeds and at least one cosmetic ingredient chosen from antioxidants, perfumes, vitamins, thickeners, emollients, moisturisers, anti-ageing agents, lifting agents, tensing agents, plumping agents, soothing agents, anti-pollution agents, lightening or depigmenting agents, fillers, nacres and the mixtures thereof.

Also, according to a preferred object of the invention, the cosmetic composition can further comprise at least one cosmetic adjuvant chosen from the group consisting of antioxidants, emollients, moisturisers, anti-ageing agents, and the mixtures thereof.

According to the nature of the composition, one or more cosmetically acceptable excipients will be chosen from emulsifiers, polymers, surfactants, rheological agents, electrolytes, pH modifiers, antioxidants, preservatives, dyes and the mixtures thereof.

As a particular example, the cosmetic composition may comprise hydrophilic gelling agents, antioxidants, preservatives and the mixtures thereof.

Cosmetic Method

Another object of the present invention is a cosmetic method for care and/or make-up of keratin materials, in particular the skin and/or lips, and notably the skin of the face and/or the neck, comprising the topical application on said keratin materials of at least one layer of the fermented extract of Longoza seeds or of a cosmetic composition containing such, as described according to the invention.

The cosmetic method of the present invention is intended, in particular, to promote and/or stimulate epidermal renewal, desquamation of the skin, vitality of the skin cells, and/or cohesion of the skin cells, and/or to prevent and/or reduce the signs of skin ageing, in particular loss of firmness, loss of elasticity, loss of density, appearance of wrinkles and/or fine lines, dryness of the skin, alteration of the skin's barrier function, alteration of the skin's suppleness and/or alteration of the homogeneity or radiance of the complexion.

The term “to promote and/or stimulate epidermal renewal” shall mean that the fermented extract of Longoza seeds is intended to promote keratinocyte differentiation.

The term “to promote and/or stimulate the desquamation of the skin” shall mean, in particular, that the fermented extract of Longoza seeds is intended to promote the renewal and development of skin cells and tissues, in particular of the skin and/or lips.

The term “to promote and/or stimulate the vitality of skin cells” shall mean, in particular, that the fermented extract of Longoza seeds is intended to promote the general condition of the skin, its radiance.

The term “to prevent and/or reduce the signs of skin ageing” shall mean, in particular, that the fermented extract of Longoza seeds is intended to reduce and/or slow the loss of firmness, loss of elasticity, loss of density, dryness of the skin, loss of suppleness, and/or appearance of wrinkles and/or fine lines.

The term “to prevent and/or reduce the alteration of the homogeneity or radiance of the complexion” shall mean, in particular, that the fermented extract of Longoza seeds is intended to reduce and/or slow the appearance of imperfections or microreliefs of the skin, rings, a cloudy, dull and/or non-uniform complexion.

The term “to prevent and/or reduce the alteration of the barrier function of the skin” shall mean, in particular, that the fermented extract of Longoza seeds is intended to reduce and/or slow changes in the integrity of the skin, which can be associated, for example, with changes in the interactions between the cells and/or between cells and the extracellular matrix, that are able to generate an alteration of the various functions of the cutaneous barrier (e.g. mechanical, moisture, antioxidant, function etc.).

According to a particular embodiment, said fermented extract of Longoza seeds according to the invention, or said cosmetic composition containing same, is applied on a skin that is aged or showing signs of ageing and/or a skin exposed to a stress, preferably a skin that is aged or showing signs of ageing.

The term “skin exposed to a stress” shall mean that the skin is subjected to a stress of endogenous or exogenous origin, in particular a mechanical or chemical stress that is able to induce non-pathological, unsightly skin reactions; for example, the skin is visibly tired and/or more sensitive to stimuli.

The skin and lips on which the compositions of the invention are applied, are healthy, i.e. not exhibiting diseases or disorders which would be part of a pathological condition (“unhealthy” subjects, affected by pathology).

Advantageously, the compositions comprising the fermented extract of Longoza seeds employed in the context of the cosmetic method of the invention are those described above.

Use of the Fermented Extract of Longoza Seeds

Another object of the invention relates to the cosmetic use of a fermented extract of Longoza seeds according to the invention for a topical application on keratin materials, in particular the skin and/or lips and in particular the skin of the face and/or neck, as a cosmetic active ingredient intended to promote and/or stimulate epidermal renewal, desquamation of the skin, vitality of the skin cells and/or cohesion of the skin cells, and/or to prevent and/or reduce the signs of skin ageing, in particular loss of firmness, loss of elasticity, loss of density, appearance of wrinkles and/or fine lines, dryness of the skin, alteration of the skin's barrier function, alteration of the skin's suppleness and/or alteration of the homogeneity or radiance of the complexion.

### EXAMPLES

The invention is illustrated by the following non-limiting examples. The percentages are expressed by weight of raw material compared with the total weight of the composition, unless otherwise indicated.

### Example 1: Method for Fermenting Longoza Seeds

Materials and Methods

Consortium of Microorganisms

The microorganisms making up the consortium comprise:
at least one lactic acid bacterium belonging to the genus Lactobacillus or Pediococcus,
at least one yeast belonging to the genus Saccharomyces, Schyzosaccharomyces or Torulaspora, and
at least one acetic acid bacterium belonging to the genus Acetobacter, Gluconobacter or Komagataeibacter.

Preparing Longoza Seeds

Raw seeds of Longoza are ground in order to obtain a powder composed of particles having an average size of approximately 250 to 500 μm.

Preparing the Nutritive Medium

The tea is prepared from 5 g/l to 10 g/l of tea leaves infused for 10 to 60 minutes at 70° C. to 90° C. Sucrose is then dissolved in the aqueous phase at a concentration between 20 g/l and 100 g/l, in general 50 g/l. The sucrose can notably be replaced by a mixture of glucose and fructose.

Fermentation Method

A fermentation of Longoza seeds was carried out according to a method comprising the following steps:
a) incubating a consortium of microorganism in a solution comprising a carbohydrate and an aqueous extract of black tea leaves (otherwise termed the “nutritive medium”) at 26° C. for 3 to 4 days in order to obtain a culture;
b) grinding Longoza seeds in order to obtain a fine powder, as described above;
c) adding, to a fermentation medium comprising dechlorinated water and a carbohydrate:
   the powder of Longoza seeds at a concentration of 1.2% or 5% by weight compared with the litre of fermentation medium, and
   the culture obtained in step a) at a concentration of 5% by weigh, compared with the weight of fermentation medium, in order to obtain a fermentation mixture;
d) incubating the fermentation mixture obtained in step c) in a fermenter at 26° C. for 10 days;
e) filtering the fermentation mixture by coarse sieving followed by filtration on a cellulose filter having an average pore size of 0.2 μm in order to obtain a fermented extract of Longoza seeds, and,
f) adding glycerin to the fermented extract of Longoza seeds at a final concentration of 70% by weight compared with the total weight.

Results

A fermented extract of Longoza seeds is obtained in the form of a solution at a concentration of 1.2 to 1.35% by weight of active material of fermented Longoza seeds, 70% glycerin and 28.65 to 28.8% water. Alternatively, a fermented extract of Longoza seeds is obtained in the form of a solution at a concentration of 1.35 to 1.65% by weight of active material of fermented Longoza seeds, 70% glycerin and 28.35 to 28.65% water.

The fermented extract of Longoza seeds obtained from a concentration of Longoza seeds of 1.2% by weight compared with the litre of fermentation medium (i.e. 12 g/L) in step c) is referred to hereafter as extract “L3”.

The fermented extract of Longoza seeds obtained from a concentration of Longoza seeds of 5% by weight compared with the litre of fermentation medium (i.e. 50 g/L) in step c) is referred to hereafter as extract “L2”.

The effect of these extracts will be illustrated in the following examples.

### Example 2: Characterisation of a Fermented Extract of Longoza Seeds

Materials and Methods

Quantification of Organic Acids by HPLC Analysis

The assay was carried out on an Agilenet 1260 DAD MS HPLC.

The extract is injected at 0.02%, diluted to 1/50^{th} in 0.1% HCOOH (weighting of 20 μL quantity sufficient for 1 mL)

The parameters in the acquisition method cited above are as follows:
Injection volume: 5 μL
Temperature: 30° C.
Column: Phenomenex Hydro-RP 150×4.6 mm 4 μm (no. 8)
Solvents:
   Isocratic 100% A: H₂O+0.1% formic acid
   Flow rate: 0.400 mL/min
   Duration of analysis 25 min

NMR

Centrifugal Partition Chromatography (CPC)

The fermented extract of Longoza seeds was lyophilised in order to reduce the quantity of water. The mass injected was approximately 4 g.

After equilibration of the column, the fermented extract of Longoza seeds was dissolved in 11 ml of lower phase+6 ml of upper phase and injected into the CPC column (303 mL column, instrument FCPE300@ (Rousselet Robatel Kromaton)) via a 20 ml sample loop. The mobile phase was pumped in the ascending mode for 65 minutes. The column was extruded by switching the mode selector valve for 10 minutes. The flow rate was 20 mL/min and the speed of rotation of the column was 1300 rpm. The two-phase solvent system corresponds to ethyl acetate/acetonitrile/water (3/3/4, v/v). The stationary phase corresponds to the lower phase of the two-phase solvent system (ascending mode) while the mobile phase corresponds to the upper phase of the two-phase solvent system.

Fractions of 20 ml were collected throughout the experiment (elution and extrusion) and combined according to their thin-layer chromatography profiles. Thus, 10 fractions were obtained.

Identification of the Main Metabolites.

An aliquot of each fraction F1 to F10 was dissolved in 700 μL of DMSO-d6 and analysed by ¹³C NMR at 298 K on a Bruker Avance AVIII-600 spectrometer (Karlsruhe, Germany) equipped with a cryoprobe. After processing the spectra, the absolute intensities of all the ¹³C NMR signals were collected automatically and grouped in the spectra of the series of fractions, using a computer script developed by Natexplore. The resulting table was subjected to a hierarchical group analysis (PermutMatrix, 1.9.3, LIRMM, Montpellier, France). The resulting 13C NMR chemical shift clusters were viewed in the form of dendrograms on a two-dimensional map. In order to identify metabolites, each ¹³C NMR chemical shift cluster obtained by HCA was manually submitted to the structure search engine of the ACD/NMR Workbook Suite 2012 (ACD/Labs, Ontario, Canada) database management software comprising the structures and predicted chemical shifts for low molecular weight natural products. 2D NMR experiments (HSQC, HMBC and COSY) were carried out on fractions containing supposedly identified compounds in order to confirm the molecular structures proposed by the database at the end of the dereplication process.

Results

The fermentation method generates a fermented extract of Longoza seeds having a very different composition from non-fermented extracts of Longoza, for example, an aqueous extract of non-fermented Longoza extracted with 100% water.

In particular, the fermented extract of Longoza seeds comprises various organic acids, as shown in Table 1 below.

Table 1: Organic Acids Present in the Fermented Extract of Seeds of Aframomum angustifolium or Longoza Compared with an Aqueous Extract of Longoza (Non-Fermented) (in Mg Compared with g of Dry Extract)

TABLE 1

| | Acetic | Citric | Gluconic | Lactic | Malic | Quinic | Succinic |
|---|---|---|---|---|---|---|---|
| | acid | acid | acid | acid | acid | acid | acid |
| | mg/g | mg/g | mg/g | mg/g | mg/g | mg/g | mg/g |
| Fermented extract of | 164 | 3.6 | 139 | 0.2 | 0.7 | 1.0 | 1.7 |
| Longoza seeds (L2) | | | | | | | |
| Fermented extract of | 80 | Nd | 9.9 | 0.6 | 0.7 | 4.4 | 4.4 |
| Longoza seeds(L3) | | | | | | | |
| Aqueous extract of | 1.9 | 17.3 | 4.2 | 0.6 | 3.0 | nd | 1.7 |
| Longoza | | | | | | | |

nd: not detected

### Example 3: Effect of the Fermented Extract of Longoza Seeds on the Expression of Skin Proteins

The effect of the fermented extract of Longoza seeds on the expression of various protein markers of the skin barrier function, cohesion of the epidermis, elasticity of the skin and radiance, was evaluated.

Materials and Methods

Culture and Treatment of NHK

Normal Human Keratinocytes (NHK) are seeded in TPP150 flasks, in supplemented Epilife medium. The NHK are trypsined at pre-confluence (0.05% Trypsin-EDTA, GIBCO-Invitrogen), and neutralised with a medium containing serum. They are then seeded in 96-well imaging plates (μ-plate 96 well, ibiTreat, Ibidi) with 10,000 cells per well.

At confluence, the NHK are treated with the fermented extract of Longoza seeds L2 or L3 in the complete Epilife medium containing 500 μM of CaCl₂ (0.2 mL/well). The L2 and/or L3 extract was tested at a concentration of 0.02% and/or 0.1% v/v. The treatment is carried out for 3 or 5 days with a replacement of the culture medium after 48 hours. For the control (untreated) conditions, the medium is replaced by a new medium containing 500 μM of CaCl₂.

Marking the Cells

At the end of the treatments, the cells are fixed with formalin (10% Mutual Buffered Formalin Solution, Sigma) for 10 minutes then made permeable with a 0.1% PBS-Triton solution (Triton X-100, Sigma). They are then saturated with 1% PBS/BSA (PBS: phosphate-buffered saline solution; BSA: bovine serum albumin), for 30 minutes at ambient temperature. The 1% PBS/BSA solution is then replaced by a primary antibody solution corresponding to each protein marked (see table 2) diluted in the 1% PBS/BSA. The plates are incubated overnight at 4° C.

After rinsing with PBS, the cells are covered with a secondary antibody solution (anti-rabbit or anti-mouse goat antibodies, Alexa Fluor 568 from Invitrogen) according to the primary antibody to be targeted at 1/300^{th} and DAPI at 1/500^{th} (4′,6′-diamidino-2-phenylindole, dihydrochloride, Invitrogen Molecular Probes) diluted in 1% PBS/BSA. The plates are incubated for one hour in the dark at ambient temperature.

After rinsing with PBS followed by distilled water, the PBS is deposited in each well in an amount of 1 mL. The plates are kept at 4° C. in the dark until acquisition of the images.

Table 2: Summary of the Primary Antibodies Used

TABLE 2

| | Primary antibodies |
|---|---|
| Loricrin | Rabbit IgG polyclonal antibodies (PAS-34945 from |
| | ThermoFisher Scientific), at 1/1000^{th} |
| Filaggrin | Mouse IgG1 monoclonal antibodies FLG01 (MA5-13440 |
| | from ThermoFisher Scientific), at 1/500^{th} |
| Elafin | Mouse IgG1 monoclonal antibodies TRAB2O (HM2062 |
| | from Hycult Biotech), at 1/200^{th} |
| Desmoglein 1 | Mouse IgG1 monoclonal antibodies 27B2 (32-6000 |
| | from ThermoFisher Scientific), at 1/200^{th} |
| Kallicrein 5 | Rabbit IgG polyclonal antibodies (ab176299 from |
| | Abcam), at 1/200^{th} |
| Transgluta- | Rabbit IgG polyclonal antibodies (PAS-59088 from |
| minase-1 | Invitrogen), at 1/500^{th} |
| ZO-1 | Mouse IgG1 monoclonal antibodies ZO1-1A12 (33-9100 |
| | from Invitrogen), at 1/500^{th} |

Image Acquisition by HCS and Analysis

The plates are scanned with the “ArrayScan XTi” from Thermo Cellomics.

Acquisition Conditions:

Detection:
DAPI: filter XF53_386_23
Alexa Fluor 568: filter XF53_572_15
Resolution: 1104×1104
Lens: 10× dry
Number of images: 25 per well

The images were analysed using the “Spot detector” image analysis software, bio-which detects the red marking of the targeted protein corresponding to its expression. The surface of the measurement zone is defined as being the entire surface of the image. The number of cells is determined by counting cores by detecting blue marking.

According to the markers and their cellular expression, the results can be expressed by:
Intensity of the marking detected (threshold)/number of cells
Intensity of the marking in the total field/number of cells

The significance of the various ratios observed between the controls and the treated samples is measured by the Student test (*p<0.05, **p<0.01).

Results

3.1. Expression of Loricrin

Loricrin is a structural protein expressed at the horny layer which contributes to the barrier function of the skin.

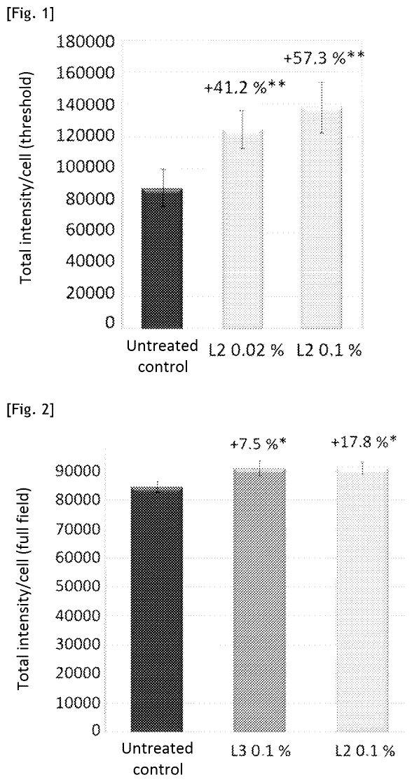

The fermented extract of Longoza seeds significantly increases the expression of loricrin with a dose effect observed when the concentration of extract is increased (+41% for extract L2 at a concentration of 0.02% and +57% for extract L2 at a concentration of 0.1%, p<0.01, see FIG. 1).

3.2. Expression of Filaggrin

Like loricrin, filaggrin is a protein involved in the barrier function of the skin.

The fermented extract of Longoza seeds significantly increases the expression of filaggrin with a dose effect observed when the concentration of extract is increased (+7.5% for extract L3 at a concentration of 0.1% and +17.8% for extract L2 at a concentration of 0.1%, p<0.01, see FIG. 2).

3.3. Expression of Elafin

Elafin is a protein involved in the skin barrier function. It also has an inhibitor effect on elastase, thus preventing the degradation of elastic fibres.

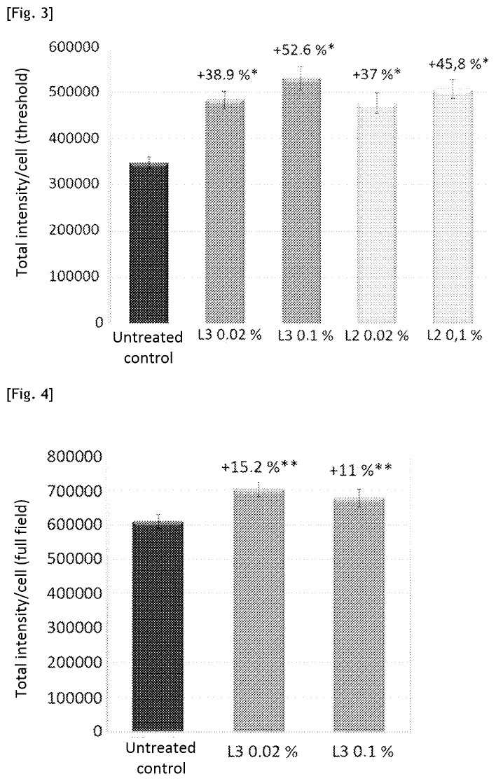

The fermented extract of Longoza seeds significantly increases the expression of elafin, with a dose effect observed when the concentration of extract is increased. Indeed, the expression is increased by +38.9% for extract L3 at a concentration of 0.02% and +52.6% for extract L3 at a concentration of 0.1% (p<0.05, see FIG. 3). Similarly, the expression is increased by +37% for extract L2 at a concentration of 0.02% and +45.8% for extract L2 at a concentration of 0.1% (p<0.05, see FIG. 3).

3.4. Expression of Kallikrein 5

Kallikrein 5 is a protein involved in homeostasis and desquamation of the skin. It also contributes to the radiance of the skin.

The fermented extract of Longoza seeds significantly increases the expression of kallikrein 5 (+15.2% for extract L3 at a concentration of 0.02% and +11% for extract L3 at a concentration of 0.1%, p<0.05, see FIG. 4).

3.5. Expression of Desmoglein 1

Desmoglein 1 is an essential protein for the integrity of the epidermis, providing the structure thereof through its adhesive function within the intracellular junctions (desmosomes). It also promotes the differentiation of keratinocytes.

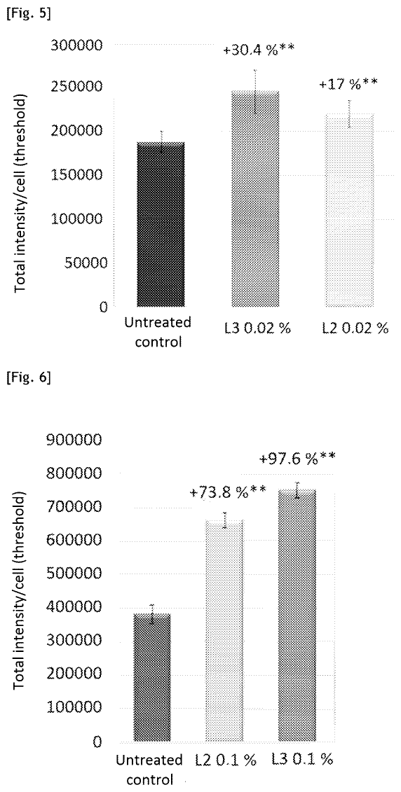

The fermented extract of Longoza seeds significantly increases the expression of desmoglein 1 (+30.4% for extract L3 at a concentration of 0.02% and +17% for extract L2 at a concentration of 0.02%, p<0.01, see FIG. 5).

3.6. Expression of Transglutaminase

Transglutaminase is an enzyme involved in the cross-linking of structural proteins in order to form the horny layer in the epidermis. It thus contributes to the barrier function of the skin.

The fermented extract of Longoza seeds significantly increases the expression of transglutaminase (+73.8% for extract L2 at a concentration of 0.1% and +97.6% for extract L3 at a concentration of 0.1%, p<0.01, see FIG. 6).

3.7. Expression of ZO-1

ZO-1 is an intracellular protein involved in the formation of tight junctions between the cells of the skin. It thus contributes to the barrier function and the integrity of the skin.

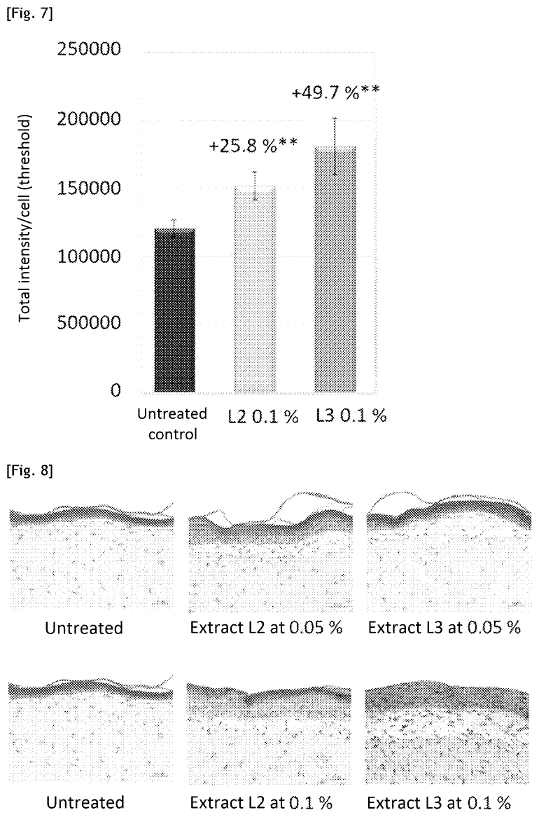

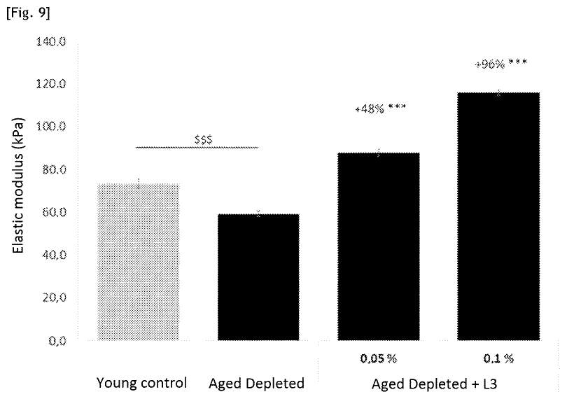

The fermented extract of Longoza seeds significantly increases the expression of ZO-1 (+25.8% for extract L2 at a concentration of 0.1% and +49.7% for extract L3 at a concentration of 0.1%, p<0.01, see FIG. 7).

### CONCLUSIONS

Two fermented extracts de Longoza seeds, L2 and L3, have been tested on the expression of markers of epidermal differentiation on a model of normal human keratinocytes in culture, in particular in order to determine their activity on the cutaneous barrier.

The extracts have positive effects on the expression of markers of the cutaneous barrier, such as elafin, involved in the constitution of the horny layer or desmoglein 1 which is a constituent of the desmosomal junctions. Furthermore, the extract L3 increases the expression of kallikrein 5, involved in homeostasis and desquamation of the skin.

### Example 4: Effect of the Fermented Extract of Longoza Seeds on a Skin Model

The effect of the fermented extract of Longoza seeds according to the present invention on the elasticity, firmness, and cellular density of the skin has been evaluated in a stem cell-depleted skin model.

Materials and Methods

Establishing the Reconstructed, Bio-Printed, Epidermal Stem Cell-Depleted (ESC), Skin Model

Bio-Printing of Papillary and Reticular Dermises by Extrusion

The powders entering into the composition of the bio-ink were dissolved in calcium-free DMEM and stirred overnight at 37° C.

The next day, young or aged, papillary/reticular fibroblasts, at confluence, were trypsined and counted. The necessary quantity of cells was sampled, for 250,000 fibroblasts per millilitre of ink prepared, then centrifuged. The cell pellet was suspended directly in the appropriate volume of bio-ink. Then, the bio-ink/cells mixture was transferred in a sterile syringe mounted on a 20-gauge cannula.

The printed objects were modelled using the Slic3r (GNU Affero General Public License) and Repetier (Hot-World GmbH & Co. KG) software. The layers of papillary dermis and reticular dermis were printed in 2 stages, one on top of the other. “Aged” dermis and “young” dermis were printed

In a first stage, cubes of dimensions 1.5 cm×1.5 cm×0.2 cm were printed using inks containing the papillary fibroblasts. The height of these objects corresponded to two superimposed layers. Following this first printing, cubes of dimensions 1.5 cm×1.5 cm×0.1 cm (equivalent to 1 layer) were printed above the first 2-layer cubes. These structures were then placed for 1 hour in a solution of calcium and thrombin, enabling the polymerisation and consolidation of the bio-ink. After rinsing three times in a saline buffer, the equivalent dermises were cultured in MCF (internal preparation of LabSkin Creations, medium suitable for culture of fibroblasts) and placed in the incubator (37° C., 5% CO₂) then cultivated for 7 days before seeding the sorted keratinocytes at their surface. The culture medium was renewed every 2 days.

Magnetic Sorting According to CD71 Marker on Primary Keratinocytes in Culture

First, adult primary keratinocytes with thawed at passage 1 in culture flasks in the culture medium MCK (internal preparation of LabSkin Creations, medium suitable for culture of keratinocytes). After one week of amplification, at confluence, the cells were trypsined then counted. All of these cells were sorted using the magnetic beads kit CD71 130-046-201 (Miltenyi) according to the manufacturer's instructions.

Briefly, the keratinocytes were incubated for 15 minutes at +4° C. with magnetic beads with a defined ratio of cells/volume of beads. This mixture was then centrifuged and the supernatant was then removed in order to take out the magnetic beads which were not bonded to the cells of interest. The pellet of keratinocytes was suspended in the culture medium, and was passed over the magnetic columns installed on the magnet provided for this purpose. The negative fraction which contains the cells not expressing the surface marker CD71, corresponding to a population enriched with epidermal stem cells not retained in the column, was harvested first in a sterile tube.

Next, the column was unhooked from the magnetic support and rinsed, making it possible to harvest the cells expressing the surface marker CD71 (stem cell-depleted fraction) which was bonded to the magnetic beads. The fractions that were enriched and depleted in cells were counted in order to calculate the ratio CD71−/CD71+.

In parallel with this extraction, previously cultured prepuce keratinocytes were also trypsined and counted in order to be seeded on the dermis the same day. These formed the “young control”.

Seeding the Keratinocytes CD71+ on the Bio-Printed Dermises

Once the magnetic sorting had been carried out and the various fractions counted, the culture medium for the “young” and “aged” bio-printed dermises was removed.

The equivalent of 500,000 keratinocytes CD71+ was deposited at the surface of the aged dermises. The same was done with a suspension of prepuce keratinocytes, seeded at the surface of the “young control” dermises, acting as reference.

After 30 minutes adhesion at +37° C., the medium was used to submerge the dermoepidermal assemblies.

The dermoepidermal assemblies were cultivated for 3 days in the MCK medium enabling proliferation of keratinocytes. They were then raised at the air/liquid interface in order to initiate the differentiation of the epidermises. This final culture step lasted for one week, in order to obtain epidermises that were multi-layered, but not completely mature.

The extract L2 or L3 was added to the culture medium. The medium was then filtered in order to avoid any risk of contamination. The extracts were then applied to the cell cultures daily for the last 5 days of culture (cultures stopped at day 35).

Samples were fixed in 4% buffered formalin solution (Alphapath, France) and then embedded in paraffin.

Histological Staining with Hematoxylin Phloxine Saffron

5 μM paraffin sections were prepared. After paraffin removal and rehydration, the samples were stained with Hematoxylin-Phloxine-Saffron (HPS).

Immunofluorescence on Sections Embedded in Paraffin

An anti-integrin alpha 6 marking was produced on sections of 5 and 20 micrometre thickness. After unmasking with heat, a step of saturation where 4% PBS/BSA blocked the aspecific antigenic sites. The cells were then incubated overnight with a specific primary antibody of the integrin alpha 6 antigen at 1/10000^{th} (rabbit monoclonal antibody, Abcam) then with a secondary antibody Alexa-Fluor 568 anti-rabbit diluted to 1/1000th (Thermo Fisher Scientific, MA, USA) and Hoechst nuclear stain (Thermo Fisher Scientific). A negative control without primary antibodies was produced in parallel.

Acquisition of Images

The histological stains and immunohistological marking were observed using the optical microscope system, Axio Observer D1/high resolution camera Axiocam (Zeiss, The Pecq, France). The images were acquired using the ZEN pro 2012 software and saved in “tif” format (high-resolution).

Acquisition of Atomic Force Microscope (AFM) Data

The AFM used is a Resolve bioscope (Bruker) on which an epifluorescence microscope (Leica DMi8) has been mounted, making it possible to carry out correlated studies (AFM image/fluorescence).

The PeakForce® QNM/Fast-Force volume mode was used. The AFM probe chosen had a theoretical stiffness constant of 0.4 N/m and radius of curvature <10 nm. Before each use, the deflection sensitivity of the probe was measured on sapphire and its stiffness constant was also calibrated using the thermal noise method. The lever of the AFM was positioned on the dermoepidermal junction (EDJ) visible due to the anti-integrin alpha 6 immunomarking carried out on frozen sections.

A complete AFM analysis consisted of the acquisition of a force volume of the zone of interest (EDJ) under aqueous conditions (PBS 1X).

The parameters used for the acquisitions were:
Force/volume matrix: 20 μm×20 μm−64p×2
Force curves: Force=20 nN−Ramp size=10 μM

For the AFM imaging of the dermis, the Force-Volume mode was also used. Force-volumes were required on various areas of the sections, with the same parameters as previously.

Quantitative Analyses: Extraction of the Elastic Modulus (Ea)

Quantifications of Ea from the raw force curves were carried out using the BioMeca Analysis (BioMeca) processing software.

The raw approach curves obtained for each section were processed to extract a precise quantification of Ea (rigidity) of the EDJ. Here, the Sneddon theoretical model was applied to an indentation between 0 and 1 μM. Using various force matrices, the variations in elasticity of the dermoepidermal junction were extracted for each section. Statistical analyses: Young control versus Aged depleted untreated CSE: t-test, $$$ p<0.001; Aged depleted untreated CSE versus Aged depleted CSE treated with L3 at 0.05% or 0.1%: t-test, ***p<0.001.

Results

4.1. Histological Analysis

Treatment with the fermented extract of Longoza seeds (L2 or L3, at a concentration of 0.05 or 0.1%) has enabled cellularised dermises to be obtained having a larger layer of newly synthesised fibroblasts than that observed in the untreated samples. The epidermises also had an improved structure: they appeared thicker, with a very clear basal organisation and a structured EDJ. The differentiation of this compartment was not disturbed by the administration of the extract—quite the contrary, a horny layer is present under all conditions. Finally, the fermented extract of Longoza seeds L3 appears to show a dose effect with a thicker layer of fibroblasts under the dermoepidermal junction with the dose at 0.1%.

4.2. Immunomarking

An analysis of the profile for expression of integrin apha-6 enabled a more precise study of the basal part of the epidermis containing the populations of epidermal stem cells. The reconstructed ESC-depleted skins treated with the fermented extract of Longoza seeds L3, at a concentration of 0.05 or 0.1%, saw their expression of integrin alpha 6 broadly increased, revealing larger populations of epidermal stem cells, firmly anchored at the dermoepidermal junction. The levels of expression of this marker reach levels comparable to, or even higher than, that observed in samples of reconstructed “young” skin.

4.3. AFM

An analysis of the modulus of elasticity of the EDJ obtained under each of the treatment conditions by AFM enabled the above cited results to be confirmed (see FIG. 9). The treatment with extract L3 at 0.05 or 0.1% led to an increase in the population of epidermal stem cells and hence to an increase in the modulus of elasticity (L3 at 0.05%: +48%, p<0.001; L3 at 0.1%: +96%, p<0.001), measured at the EDJ, indicating that these junctions are more rigid. Furthermore, the fermented extract of Longoza seeds has shown a dose effect, the module of elasticity increasing with the concentration of active ingredient applied in the culture medium.

Also, the fermented extract of Longoza seeds L3, by introducing an increase in the proliferative compartment at the basal level of the epidermis, enables a more rigid and a more strongly anchored EDJ to be obtained, as well as thicker and more proliferative epidermises and which however have a satisfactory differentiation.

### Example 5: Cosmetic Formulations

The fermented extract of Longoza seeds such as prepared in example 1, is formulated in the following illustrative and non-limiting compositions, prepared according to conventional formulation methods in cosmetics.

5.1. Composition in the Form of a Lotion

TABLE 3

| Ingredients | % by weight |
|---|---|
| Glycerin | 6.0000 |
| Butylene glycol | 5.0000 |
| Cetyl alcohol | 1.0000 |
| Hydrogenated polyisobutene | 9.000 |
| Fermented Longoza* | 0.2000 |
| Perfume, neutralisers, preservatives | q.s. |
| Purified water | q.s. 100% |

*prepared according to example 1

Applied on the skin, in particular on the face, this composition provides an anti-ageing action with beneficial effects on the firmness and/or the elasticity of the skin.

5.2. Composition in the Form of a Serum

TABLE 4

| Ingredients (INCI NAME) | % by weight |
|---|---|
| HYDROGENATED POLYISOBUTENE | 8.0000 |
| CAPRYLIC/CAPRIC TRIGLYCERIDE | 4.0000 |
| GLYCERYL STEARATE | 1.5000 |
| PEG-100 STEARATE | 2.5000 |
| CERA ALBA | 1.5000 |
| CETEARYL ISONONANOATE | 1.5000 |
| CETYL ALCOHOL | 1.0000 |
| STEARYL ALCOHOL | 1.6000 |
| DIMETHICONE | 2.0000 |
| PHENOXYETHANOL | 1.7000 |
| FERMENTED LONGOZA* | 0.5000 |
| CAPRYLYL GLYCOL | 0.3000 |
| XANTHAN GUM | 0.1800 |
| ACRYLATES/C10-30 ALKYL ACRYLATE | 0.1500 |
| CROSSPOLYMER | |
| SODIUM HYDROXIDE | 0.0300 |
| TOCOPHEROL | 0.0004 |
| PERFUME, NEUTRALISER, PRESERVATIVE | q.s. |
| PURIFIED WATER | q.s. 100% |

*prepared according to example 1

The serum is applied on all of the face and in particular on areas showing signs of ageing. The serum comprising the fermented extract of Longoza seeds according to the invention promotes epidermal renewal, for an elastic skin that is firmer and a more homogeneous complexion.

1-13. (canceled)

14. A fermented extract of Aframomum angustifolium or Longoza seeds, comprising:
a quinic acid content of 0.05% to 2%; and
an acetic acid content of 5% to 15%,
by weight compared with the weight of dry extract of the fermented extract.

15. The fermented extract of Aframomum angustifolium or Longoza seeds, according to claim 14, wherein it is in liquid form and comprises at least one preservative.

16. The fermented extract of Aframomum angustifolium or Longoza seeds, according to claim 15, wherein the preservative(s) are selected from butylene glycol, glycerin, propylene glycol, sodium benzoate benzoate, and potassium sorbate.

17. A method for fermenting Aframomum angustifolium or Longoza seeds, comprising the following steps:
a) incubating a consortium of microorganisms in a solution comprising a carbohydrate at a temperature of 17° C. to 38° C. for 1 to 10 days in order to obtain a culture of microorganisms,
said consortium comprising at least one lactic acid bacterium belonging to the genus Lactobacillus or Pediococcus, at least one yeast belonging to the genus Saccharomyces, Schyzosaccharomyces, or Torulaspora, and at least one acetic acid bacterium belonging to the genus Acetobacter, Gluconobacter, or Komagataeibacter;
b) grinding the Aframomum angustifolium or Longoza seeds, in order to obtain a powder composed of particles having an average size of 100 μm to 1 mm;
c) adding said powder to a fermentation medium comprising the culture of microorganisms in order to obtain a fermentation mixture,
said fermentation medium comprising a carbohydrate chosen from sucrose, glucose, fructose, molasses, or a mixture of one or more thereof,
d) incubating the fermentation mixture at a temperature of 25° C. to 35° C. for 7 to 13 days;
e) filtering the fermentation mixture in order to obtain a fermented extract of Aframomum angustifolium or Longoza seed.

18. The method according to claim 16, wherein approximately 60 g of powder or less is added per litre of fermentation medium in step a).

19. The method according to claim 17, wherein the fermentation medium further comprises an aqueous extract of tea leaves.

20. A fermented extract of Aframomum angustifolium or Longoza seeds, obtained by the method according to claim 17.

21. A cosmetic composition comprising, in a physiologically acceptable medium, a fermented extract of Aframomum angustifolium or Longoza seeds, according to claim 14.

22. The cosmetic composition according to claim 21, wherein the fermented extract of Aframomum angustifolium or Longoza seeds is present in a percentage ranging from 0.0000012% to 0.0825%, by weight of active material (dry matter of fermented extract of Aframomum angustifolium or Longoza seeds) compared with the total weight of said composition.

23. The cosmetic composition according to claim 21, wherein it further comprises at least one cosmetic adjuvant chosen from the group consisting of antioxidants, emollients, moisturisers, anti-ageing agents, perfumes, and mixtures thereof.

24. The cosmetic composition according to claim 21, wherein it is in the form of a cream, oil-in-water emulsion, water-in-oil emulsion or multiple emulsion, solution, suspension, gel, milk, lotion, or serum.

25. A cosmetic method for care and/or make-up of keratin materials, comprising the topical application on said keratin materials, of at least one layer of a cosmetic composition as defined in claim 21.

26. The cosmetic method according to claim 25, wherein it is intended to promote and/or stimulate epidermal renewal, desquamation of the skin, vitality of the skin cells, and/or cohesion of the skin cells, and/or to prevent and/or reduce the signs of skin ageing.

27. The cosmetic method according to claim 26, wherein it is intended to prevent and/or reduce loss of firmness, loss of elasticity, loss of density, appearance of wrinkles and/or fine lines, dryness of the skin, alteration of the skin's barrier function, alteration of the skin's suppleness and/or alteration of the homogeneity or radiance of the complexion.

28. A cosmetic use of a fermented extract of Aframomum angustifolium or Longoza seeds, as defined according to claim 14, as active ingredient to promote and/or stimulate epidermal renewal, desquamation of the skin, vitality of the skin cells, and/or cohesion of the skin cells, and/or to prevent and/or reduce the signs of skin ageing.
